(19) United States
(12) Patent Application Publication
(10) Pub. No.: US 2006/0057181 A1
(43) Pub. Date: Mar. 16, 2006

(54) Method for determining the gastric evacuation using a 13c-labelled test meal

(76) Inventor: Sitke Aygen, Cologne (DE)

Correspondence Address: JACOBSON HOLMAN PLLC, 400 SEVENTH STREET N.W., SUITE 600, WASHINGTON, DC, 20004, US

(21) Appl. No.: 10/532,348

(22) PCT Filed: Oct. 24, 2003
(86) PCT No.: PCT/EP03/11787

§ 371 Date: Jul. 20, 2005

(30) Foreign Application Priority Data
Oct. 26, 2002 (DE) 102 49 929.2

(51) Int. Cl. A61F 2/00

(52) U.S. Cl. 424/423

(57) ABSTRACT

The method for determining the gastric emptying by orally administering a test substance and measuring its dwelling time in the stomach comprises determining the increase of ¹³CO₂ in the exhaled respiratory air before and after the oral administration of free ¹³C-octanoic acid together with a standardized test meal.

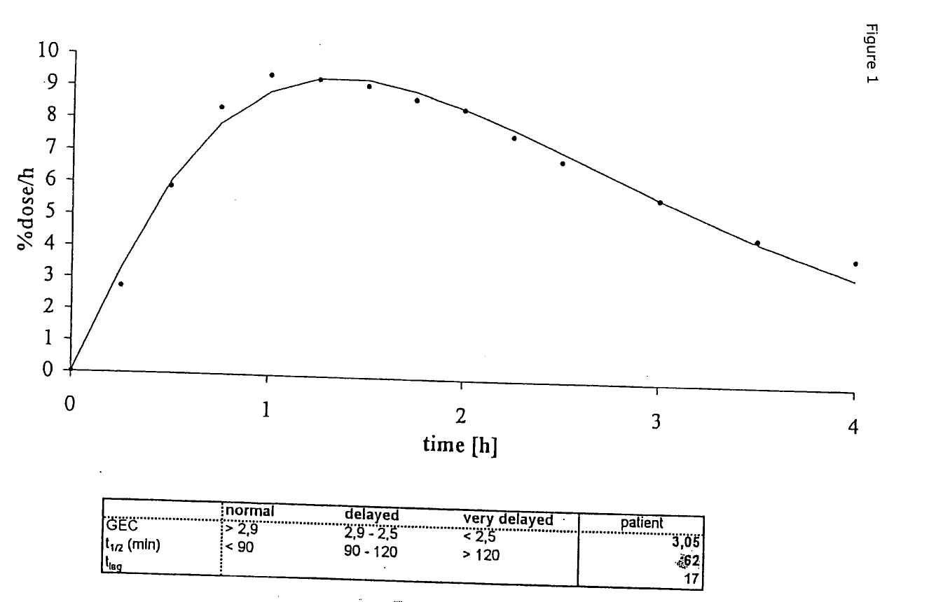

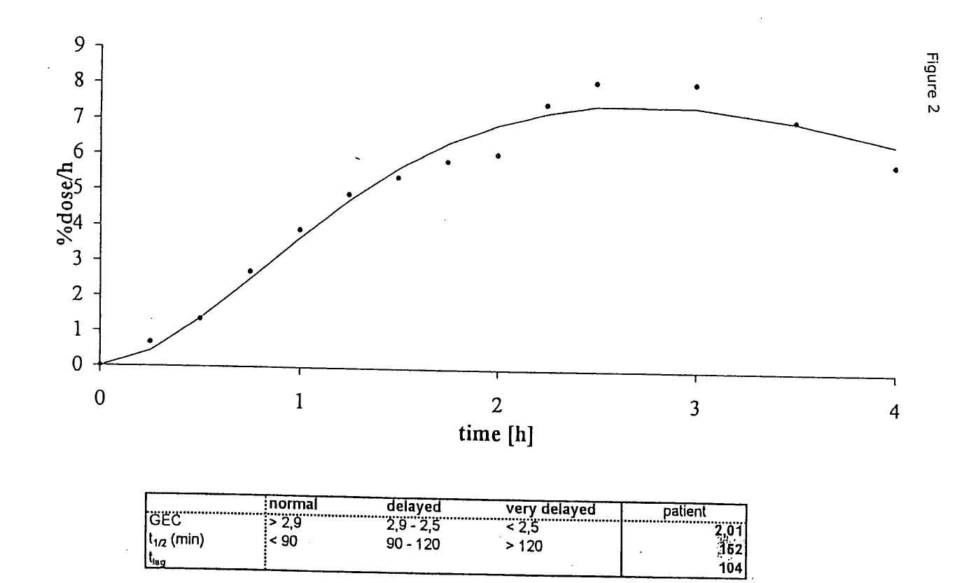

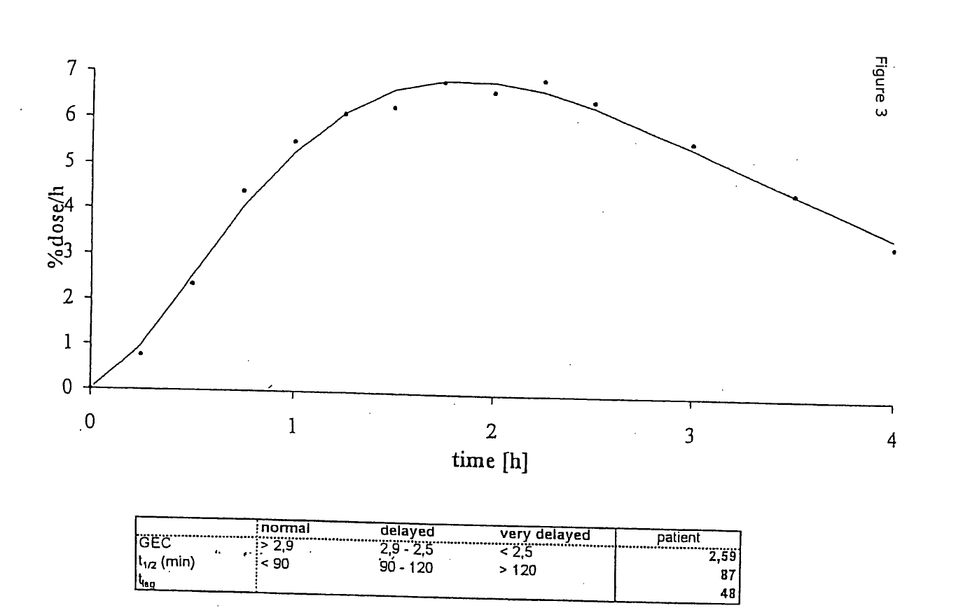

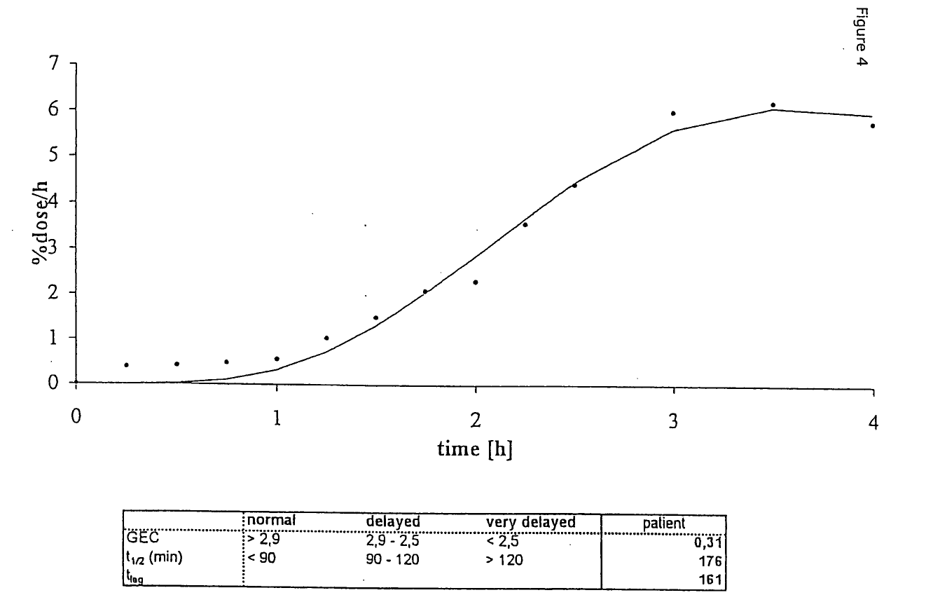

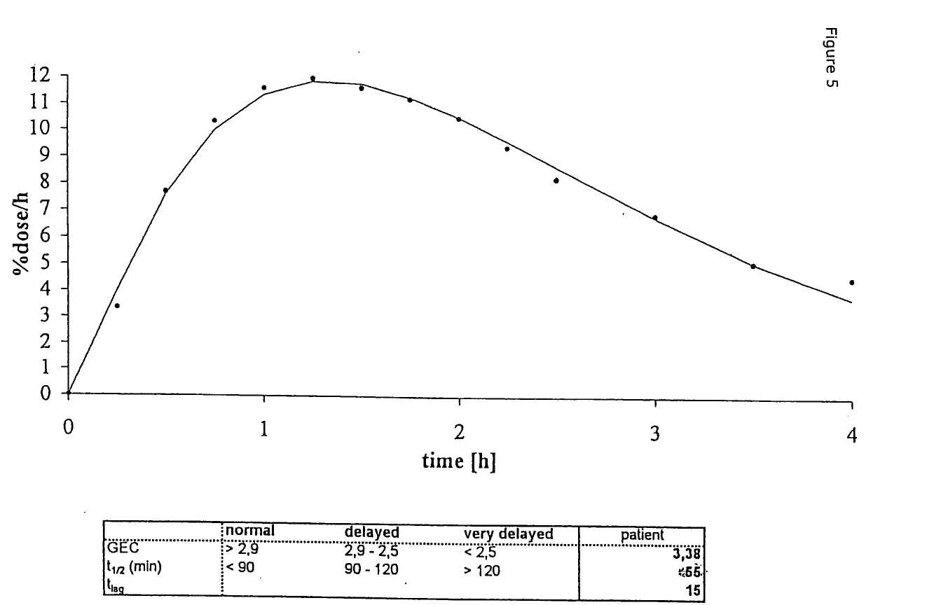

The present invention relates to a method for determining the gastric emptying by orally administering a test substance and measuring its dwelling time in the stomach.

The existing so-called “gold standard” of this determination comprises orally administering a ^{99m}Tc-protein colloid to the patient, followed by establishing the whereabouts of this radioactive isotope by radioscintigraphy.

This test results in a per se undesired radioactive load on the person to be examined, so that validation of the test results in the same person under the same conditions has been dispensed with to date. A further disadvantage of this method is the fact that the dwelling time is subject to significant variation also in healthy persons, which can be accounted for, inter alia, by the different shapes of the stomach as well. Further, greater movements of the body already result in a change of the position of the radioactive marker and thus in incorrect results.

Examinations of the rate of gastric emptying with the salt of ¹⁴C-octanoic acid and ¹³C-octanoic acid with a partial meal yielded clearly different results which also exhibited significant deviations, above all, from the values established by the “gold standard”. As the test meal, the ¹⁴C-octanoic acid or ¹³C-octanoic acid in the form of salts were stirred into an omelette batter, which was then baked; it was considered that in healthy patients such an omelette would be digested within a relatively short time to such an extent as to be absorbed in the duodenum; cf. Ghoos et al., Gastroenterologie 1993; 104: pages 1640 to 1647.

Since there is still a need for a simple determination method with a lesser load on the patient for determining the gastric emptying, further intensive examinations have been performed which, although resulting in a somewhat better correlation of the values with the “gold standard”, were by no means satisfactory.

Only several repetitions of this test with the same patient with different test meals yielded the surprising result that it is crucial that the ¹³C-octanoic acid is bound in free form to the protein of an egg yolk, and that it must not be bound in the form of a salt with an inorganic cation. However, the previous examinations with an omelette used the ¹³C-octanoic acid in the form of its sodium salt due to its simpler handling. However, it was not recognized or taken into account that only the free octanoic acid is quickly bound to egg yolk. Thus, the results with the salt are not comparable. Further, it was not recognized that the reproducibility is increased if the free ¹³C-octanoic acid is bound to egg yolk rather than egg white. In contrast, the sodium salt is water-soluble and therefore passes more quickly through the stomach.

The intensive further examinations further yielded the result that, when the free ¹³C-octanoic acid is used in a form bound to egg yolk, an optimum digestion takes place in the stomach, so that in healthy persons this test meal is passed to the duodenum within a relatively short period of time, from where the ¹³C-octanoic acid is then transported via the portal system into the liver, where it is oxidized to ¹³CO₂. The thus formed ¹³CO₂ is then measured in the exhaled respiratory air by means of IRMS. IRMS (isotope ratio mass spectrography) has in the meantime become the most sensitive and best method for determining ¹³CO₂ in the exhaled respiratory air.

Thus, the optimized test meal now consists of a fried egg into which the free ¹³C-octanoic acid is stirred, whereupon it is eaten together with a slice of toasting bread, 5 to 10 g of margarine or butter, followed by drinking 150 ml of water or coffee. The amount of ¹³C-octanoic acid is generally from 40 to 100 mg. For example, 99% enriched ¹³C-octanoic acid from Isotec, Miamsbrough, Ohio, USA, is suitable. Further, it was found that the tedious body-related conversion factors, such as body mass index, as demanded by Ghoos et al., loc. cit., can be dispensed with, because these play a substantially lesser role in the calculation of gastric emptying as compared to the variation range between normal, delayed and very delayed gastric emptying. In the method according to the invention, changing the dose only affects the height of the curve, but not the timely course of metabolization and thus the time of maximization. The attached curves and data obtained for the course of ¹³CO₂ excretion in five different patients show that for normal gastric emptying the maximum of ¹³CO₂ excretion is at >2.9 h, and the half life is at <90 min (cf. No. 1). For delayed gastric emptying, the maximum is at 2.9 to 2.5 h, and the half life is between 90 and 120 min (cf. Nos. 2 and 3). For very delayed gastric emptying, the maximum is at <2.5 h, and the half life is at >120 min (cf. No. 4). When the gastric emptying is somewhat faster, the maximum is at >2.9 h, and the half life is at <90 h (cf. No. 5). Therefore, the sampling and optimum calculation take three to four hours. The interval for sampling is optimal at 15 min.

For the same patients, these values were compared with the results of singular scintography according to Siegel and showed a correspondence which was only relatively good.

In contrast, repetitions with the same patient by the method according to the invention showed a significantly better correspondence, which is accounted for by the above explanations.

Since only with the method according to the invention it was possible and reasonable to perform the same test repeatedly with the same patient, it was established for the first time that reproducibility is very high and that the test results are thus much closer together than expected from a comparison with the “gold standard”. Thus, the previously observed data variations of results from different methods are not necessarily based on errors inherent to the methods, but rather on error sources in the “gold standard” and a non-optimized test meal.

Thus, the method according to the invention is superior to the “gold standard” in several respects: The administration of a radioactive substance is dispensed with, which is not admissible anyway for pregnant and nursing females. Further, the patient need not sit calmly between the measuring devices for radioactivity. Rather, it is sufficient if he blows samples of his respiratory air into collecting devices every 12 minutes. For example, the so-called vacutainerss are particularly suitable for this purpose. These samples of the exhaled respiratory air may then be measured and evaluated in a central laboratory with a correspondingly sensitive IRMS equipment.

Deviations from normal times of gastric emptying may give indications of a wide variety of diseases, such as gastritis, gastric carcinomas, diabetes (ketoacidosis and gastroparesis), hypothyroidism, uremia, hyperkaliemia, hypercalcemia, hepatitic coma, stomach surgery, such as postsurgical ileus, vagotomy or resection of the stomach as well as neurological disorders of the central nervous system or damage from anticholinergic or opioid drugs, tricyclic antidepressives etc. On the other hand, too fast an emptying is an indication of the Zollinger-Ellison syndrome, vagotomy and pyloroplasty/antrectomy, influence of drugs such as casapride, domperidone, metoclopramide etc. Actually, the method according to the invention is unsuitable only for diseases of the liver or pancreas and for duodenal reflux. In contrast, an advantage of the method according to the invention is the fact that it may also be employed for the follow-up of therapies due to its being repeatable without problems.

Thus, a simple and inexpensive determination method with a low load on the patient has now become available, which may also be employed more often than the previous “gold standard” for this reason.

1. A method for determining the gastric emptying by orally administering a test substance and measuring its dwelling time in the stomach, characterized in that the increase of ¹³CO₂ in the exhaled respiratory air is determined before and after the oral administration of free ¹³C-octanoic acid in a form bound to egg yolk together with a standardized test meal, wherein body-related conversion factors are dispensed with.

2. The method according to claim 1, characterized in that said test meal consists of a fried egg into which the free ¹³C-octanoic acid is stirred, whereupon It is eaten together with a slice of toasting bread, 5 to 10 g of margarine or butter, followed by drinking 150 ml of water or coffee.
